(19) United States
(12) Patent Application Publication
(10) Pub. No.: US 2005/0196789 A1
(43) Pub. Date: Sep. 8, 2005

(54) Preparation of biologically derived fluids for biomarker determination by mass spectrometry

(75) Inventors: Andrew J. Tomlinson, Wayland, MA (US); Cheryl E. Murphy, Hudson, NH (US)

Correspondence Address: APPLIED BIOSYSTEMS, 500 OLD CONNECTICUT PATH, FRAMINGHAM, MA, 01701, US

(73) Assignee: Applera Corporation, Framingham, MA (US)

(21) Appl. No.: 11/051,807

(22) Filed: Feb. 4, 2005

Related U.S. Application Data
(60) Provisional application No. 60/542,359, filed on Feb. 6, 2004.

(51) Int. Cl. C12Q 1/68

(52) U.S. Cl. 435/6

(57) ABSTRACT

Methods and kits for preparing biologically derived fluids for subsequent biomarker analysis by mass spectrometry are provided.

### CROSS REFERENCE TO RELATED APPLICATIONS

This application claims the benefit of U.S. Provisional Application No. 60/542,359, filed on Feb. 6, 2004, incorporated herein by reference.

### FIELD

This invention pertains to the field of analysis by mass spectrometry.

1. Introduction

Identification and validation of soluble proteinaceous, and small molecule biomarkers has wide utility, ranging from classification of disease and normal patient populations for disease diagnosis, to understanding the effects of therapeutics in pharmaceutical development. Such biomarkers of disease or therapeutic effects are likely to be present in fluids that are easily and with minimal intrusion collected from patients. For example, biological fluids that can be collected from a patient or patients in a clinical setting including, but are not limited to, urine, blood (plasma or serum), amniotic fluid, saliva, cerebral spinal fluid (CSF), puss or fluids from a glandular secretion. These samples can then be analyzed by techniques that could determine the presence of specific biomarkers or profiles of biomarkers that can be indicative of the population (disease or normal, treated or untreated, etc.) to which the patient belongs. In a clinical setting any method of analysis is preferably simple; using few steps and using systems that require inexperienced operators or technicians.

For example, surface enhanced laser desorption ionization time of flight mass spectrometry (SELDI-TOF-MS) can be used for screening patient samples by assessing profiles that are produced. This approach uses solid phases attached to the surface of sample targets for capturing components of a biological fluid and subsequent analysis by time of flight mass spectrometry (TOF-MS). Profiles of at least two patient populations (e.g. diseased and normal, treated and untreated, etc.) can be produced. Algorithms can be trained and used to predict the group to which unknown samples belong. Various active surfaces including hydrophobic, anion or cation exchange, and affinity media have been utilized by this technique for the identification of biomarker profiles. Surface modified chips can also prepared in parallel to ensure rapid sample analysis. However, the low resolution of collected profiles is such that identification of specific biomarkers is often not practical, and validation of individual biomarkers of disease or therapeutic treatments is not possible. Additionally, components that are not bound by the active surface can be washed away or can be lost before collection of each profile. Loss of components unbound to the active surface is likely to discard other potentially important biomarkers of the disease or treatment under investigation. Accordingly, better methods for the analysis of biomarkers from biologically derived fluids could be a useful advance in biomarker discovery and/or the identification and/or treatment of patients.

### BRIEF DESCRIPTION OF THE DRAWINGS

FIG. 1 is a Linear mode MALDI-TOF MS spectrum of a pooled normal serum prepared using a C4 solid phase support.

FIG. 2 is a Linear mode MALDI-TOF MS spectrum of a pooled normal serum prepared using a C18 solid phase support.

FIG. 3A is a Reflector mode MALDI-TOF MS spectrum of a pooled normal serum prepared using a C4 solid phase support without depletion of high molecular proteins using ultrafiltration by a 50,000 molecular cut-off filter.

FIG. 3B is a Reflector mode MALDI-TOF MS spectrum of a pooled normal serum prepared using a C4 solid phase support with depletion of high molecular proteins using ultrafiltration by a 50,000 molecular cut-off filter.

FIG. 4A is a Reflector mode MALDI-TOF MS spectrum of a pooled normal serum prepared using a C18 solid phase support without depletion of high molecular proteins using ultrafiltration by a 50,000 molecular cut-off filter.

FIG. 4B is a Reflector mode MALDI-TOF MS spectrum of a pooled normal serum prepared using a C4 solid phase support with depletion of high molecular proteins using ultrafiltration by a 50,000 molecular cut-off filter.

FIG. 5 is a Reflector mode MALDI-TOF MS spectrum of a second pooled male normal serum prepared using a C4 solid phase support with depletion of high molecular proteins using ultrafiltration by a 50,000 molecular cut-off filter.

FIG. 6A is a Reflector mode MALDI-TOF MS spectrum of a pooled normal serum prepared by dilution in a binding buffer containing 1M guanidine hydrochloride, saline, TBAP, and TFA using a C18 solid phase support with depletion of high molecular proteins using ultrafiltration by a 50,000 molecular cut-off filter.

FIG. 6B is a Reflector mode MALDI-TOF MS spectrum of a pooled normal serum prepared by collection of a second fraction from the ultrafiltration filter use to generate FIG. 6A using a C18 solid phase support. For this fraction the filter was washed with a buffer containing 2M guanidine hydrochloride, saline, TBAP, and TFA.

FIG. 7A is a Linear mode MALDI-TOF MS spectrum of a pooled normal serum prepared by dilution in a binding buffer containing 1M guanidine hydrochloride, saline, TBAP, and TFA using a C18 solid phase support with depletion of high molecular proteins using ultrafiltration by a 50,000 molecular cut-off filter.

FIG. 7B is a Linear mode MALDI-TOF MS spectrum of a pooled normal serum prepared by collection of a second fraction from the ultrafiltration filter use to generate FIG. 6A using a C18 solid phase support. For this fraction the filter was washed with a buffer containing 2M guanidine hydrochloride, saline, TBAP, and TFA

FIG. 8A is a Reflector mode MALDI-TOF MS spectrum of a pooled normal serum prepared by using a C18 solid phase support. This fraction was generated using a binding buffer containing 2M guanidine hydrochloride, saline, TBAP, and TFA.

FIG. 8B is a MALDI-TOF MS/MS spectrum for a component detected at m/z 2753 in the reflector spectrum of the pooled serum (provided in FIG. 8A). This component was identified as a peptide derived from albumin.

### DESCRIPTION OF VARIOUS EMBODIMENTS

This invention relates to an approach that can be used for identifying biological markers (e.g. biomarkers) of disease and/or therapeutic interest from biologically derived fluids (such as those from patient samples). In some embodiments, this invention relates to methods for the preparation of biologically derived fluids for subsequent analysis by mass spectrometric techniques, such as matrix-assisted laser desorption ionization time of flight mass spectrometry (MALDI-TOF-MS) as well as the determination of biological profiles and/or specific biomarkers that can be used to characterize patient samples as being derived from different populations. For example, the analysis can be used to determine normal verses diseased states or treated verses untreated states. In some embodiments, the analysis can be applied to groups and in some embodiments the analysis can be applied to individuals.

In accordance with various embodiments, methods and kits are provided to prepare biologically derived fluids for subsequent analysis by mass spectrometry, including but not limited to MALDI-TOF-MS. The methods and kits can be used for the determination of discrete biomarkers, or for profiles of biomarkers that can be classified as being derived from one of two or more patient populations. Solid phase extraction can be used in the methods and kits to adsorb and optionally fractionate components of a biological fluid. Multiplexing sample preparation can be conveniently achieved using one of several commercially available microtiter plate format solid phase sample preparation plates (such as the ZipPlate™ from Millipore). Alternatively, single samples may be prepared using micro-column devices (such as ZipTip™ from Millipore).

In some embodiments, samples of a biological fluid can be prepared for immobilization to a solid phase by dilution with a binding buffer. The binding buffer can comprise various reagents including physiological saline (or other salt for adjusting ionic strength) to ensure each patient sample is isotonic before application to the solid phase extraction device. In some embodiments, the saline concentration can be in the range of about 0.1% to about 2.0%. In some embodiments, the saline concentration can be in the range of about 0.5% to about 1.25%. In some embodiments, the saline concentration can be in the range of about 0.75% to about 0.95%. The saline concentration of the binding buffer can be used to adjust or normalize the different salt concentrations in patient derived samples.

Additionally, the binding buffer can comprise a chaotropic agent. Non-limiting examples of suitable chaotropic agents include, but are not limited to, urea, thiourea, guanidine hydrochloride or the like. In some embodiments, the concentration of the chaotropic agent can be in the range of about 0.01M to about 8 M. In some embodiments, the concentration of the chaotropic agent can be in the range from about 0.05M to about 2.5 M. The binding buffer can also comprise a reducing agent. Non-limiting examples of suitable reducing agents include, but are not limited to, dithiothreitol (DTT), dithioerythritol (DTE), 2-mercaptoethanol (BME), tributylphospine (TBP), tris(2-carboxyethyl)phosphine hydrochloride (TCEP). In some embodiments, the concentration of reducing agent can be in the range of about 0.01 mM to about 100 mM. In some embodiments, the concentration of reducing agent is in the range of about 1 mM to about 50 mM. In some embodiments, the concentration of reducing agent is in the range of about 5 mM to about 25 mM.

The binding buffer can also comprise one or more acidifying agents. Some examples of acidifying agents are volatile organic acids. Non-limiting examples of suitable volatile organic acids include, but are not limited to, formic acid, acetic acid, trifluoroacetic acid, or the like. Other such acids will be known to one of skill in the art of MALDI-TOF mass spectrometry. In some embodiments, the composition of the volatile organic acid is in the range of about 0.001% to about 5% v/v of the binding buffer solution. In some embodiments, the composition of the volatile organic acid is in the range of about 0.001% to about 1.0% v/v of the binding buffer solution.

In addition to the one or more acidifying agents, the binding buffer can comprise one or more strong ion-pair reagents. Although the volatile organic acid may itself be an ion-pair reagent, strong ion pair reagents include, but are not limited to heptafluorobutyric acid, tetrabutylammonium phosphate (TBAP), triethylamine, trifluoracetic acid or the like. In some embodiments the composition of the ion pair reagent is in the range of about 0.5 mM to about 100 mM. In some embodiments the composition of the ion pair reagent is in the range of about 0.5 mM to about 10 mM. In some embodiments the composition of the ion pair reagent is in the range of about 0.5 mM to about 2.5 mM.

The binding buffer can be used to dilute the biologically derived sample. For patient derived biological fluids, a biological sample to binding buffer volume ratio of 1 to 10, respectively, can be used to prepare the sample for solid phase adsorption and optional fractionation. Other ratios of biological sample to binding buffer can be used. Typically the binding buffer will be used in a volume that is many times (e.g. from about 5 times to about 20 times) the volume of the biological sample.

According to some embodiments, a biologically derived sample can be diluted with the binding buffer (as described above) and loaded onto a suitable solid phase device comprising a small bed (e.g. 300 nL) of a suitable solid phase (e.g. HPLC media). Suitable solid phases are known to artisans and include, but are not limited to, hydrophobic phases (e.g., C4, C8 or C18 alkyl chains, etc.), ion exchange phases (such as strong and weak cation exchange and strong and weak anion exchange media), and affinity based phases. The solid phases can comprise one or more of a plethora of bait molecules, capture specific molecular classes or can be designed to capture discrete biomolecules. Binding and elution of components of the biological sample to the solid phase extraction media can be performed by a number of approaches know to artisans. For example, when using a microtiter plate device (such as the ZipPlate™ from Millipore) sample transfer through the solid phase can be accomplished by application of low vacuum (e.g. 2-10 inches of Hg) to the exit side of the plate.

Following component adsorption from the binding buffer diluted sample, the solid phase can be washed with a suitable aqueous wash solvent to remove hydrophilic components of the binding buffer and patient sample. The washing can reduce the concentrations of saline, and other salts, chaotropic agent, reducing agent, and other low molecular weight hydrophilic components of the sample and binding buffer mixture to a level so as not to interfere with subsequent mass spectral analyses. A suitable wash solvent can be an aqueous solvent that comprises mixtures of a volatile polar organic solvent (e.g. acetonitrile, methanol or ethanol), water and volatile organic acid.

Once the washing is complete, one or more (or all) of the absorbed components can be eluted from the solid phase using a suitable elution solvent. The elution solvent can comprise the matrix required for MALDI-TOF-MS analysis (when this mass spectrometric technique is used for sample analysis) or the matrix can be added after the components of the sample have been eluted from the solid phase. A non-limiting example of an elution solvent comprises a volatile polar organic solvent, water and organic acid. Suitable matrices for MALDI-TOF-MS analysis include, but are not limited to, alpha cyano-4-hydroxy-cinnamic acid (CHCA), sinnapinic acid, 2,5-dihydroxybenzoic acid (DHB) and the like. Other matrices suitable for MALDI-TOF-MS analysis are known to one of skill in the art. In some embodiments the elution solvent can comprise an ammonium salt including but not limited to mono basic ammonium phosphate, ammonium citrate and the like to enhance MALDI performance (See: Zhu X, Papayannopoulos I A, J. Biomol. Tech. 2003; 14(4): 14). When ion exchange solid phase media are used for preparation of biological fluids, salt or salts are also required components of the elution solvent. The salt or salts include volatile salts such as ammonium chloride, ammonium acetate, ammonium formate as well as non-volatile salts such as sodium or potassium salts.

In some embodiments, desorption of the components of the biological sample from the solid phase can be collected in a single fraction. In some embodiments, desorption of the components of the biological sample from the solid phase can involve a fractionation process whereby the eluent from the solid phase device is collected in two or more fractions. For example, if a hydrophobic solid phase is used, a stepwise or linear gradient of organic modifier (e.g. acetonitrile, methanol or ethanol) can be added to the elution solvent over time to thereby effect differential desorption of the immobilized components wherein two or more fractions of the eluent from the solid support are collected. The one or more collected fractions can each undergo MS analysis.

Regardless of how performed, components of the biological sample that have been adsorbed to the solid phase and then desorbed (eluted) can be captured either directly onto the MALDI plate (when MALDI-TOF-MS is the mass spectrometric technique of choice) or they can be captured in a microtiter plate for analysis by other analytical techniques such as electrospray mass spectrometry (ESI-MS), combined liquid chromatography-electrospray-mass spectrometry (LC-ESI-MS) or combined liquid chromatography-MALDI-TOF-MS (LC-MALDI).

In some embodiments, other fractionation processes can be performed either before or after solid phase adsorption. For example, a low molecular weight fraction of the biological fluid can be prepared prior to further sample preparation using solid phase extraction methods. A low molecular weight fraction of a biologically derived fluid can be conveniently prepared before or after dilution with the binding buffer (as described above) using ultra filtration spin filters (such as microcon filters from Millipore having a molecular weight cut-off). In some embodiments, the molecular weight cut-off of the ultra filtration device can be 10-50 kDa. In some embodiments, the molecular weight cut-off of the ultra filtration device can be 20-50 kDa. In some embodiments, the molecular weight cut-off of the ultra filtration device can be 30-50 kDa.

An advantage of this approach is further fractionation of biologically derived samples with retention of the larger molecular weight fraction in the ultra filtration device. Removing the large proteins from the sample reduces ion suppression effects of many mass spectrometric techniques (particularly those known to occur in MALDI-TOF-MS processes), thereby providing a cleaner more reproducible spectrum of the lower molecular weight fraction of the biological fluid. Additionally, retention of the larger molecular weight fraction enables further analysis by digestion of the retained components of the biological sample (e.g. protein) with an enzyme (e.g. trypsin) in the ultra filtration device, collection of the digestion products (e.g. peptides) there formed, and optional further fractionation by solid phase extraction using the methods as described above. Regardless of whether or not additional processing occurs, collected materials can then be analyzed by MALDI-TOF-MS or by other suitable techniques such as ESI-MS, LC-ESI-MS, LC-MALDI, or the like.

In some embodiments, abundant proteins of the biological fluid can be depleted or removed prior to further fractionation by solid phase extraction, and may also include fractionation by ultra filtration. Depletion of serum albumin, immunoglobulins, transferrin from sera and beta-2-migroglobulin from urine usually improves the dynamic range of mass spectral analysis, enabling detection of those components of the biological fluid that are of lower abundance. Coupling this approach with the solid phase extraction, and ultra filtration methods of the current teachings serves to further simplify, and compartmentalize isolated fractions, thereby reducing ion suppression effects of mass spectrometric techniques. Ultimately, sample profiles can be simplified to thereby enable the identification of specific biomarkers, and/or classification of patients into one or more populations (e.g., diseased or normal, treated or untreated, etc.).

Following fractionation of the biologically derived fluid and analysis of said fraction by mass spectrometry, data analysis can be performed by a variety of statistical approaches known to artisans. Approaches such as principle component analysis, Wilcoxon tests, and other trained discriminating algorithms are suitable for identification of biomarkers in the profiles produced by the current teachings. Discrete biomarker identification and sample classification by profile appearance can be used to identify the population to which a specific patient sample belongs.

The following examples are provided to illustrate the presently described invention and are not intended to be limiting in any way.

### EXAMPLES

### Example 1

### Preparation of a Pooled Normal Male Serum (Sigma H-3188) Using a C4 Solid Phase with Analysis by MALDI-TOF-MS

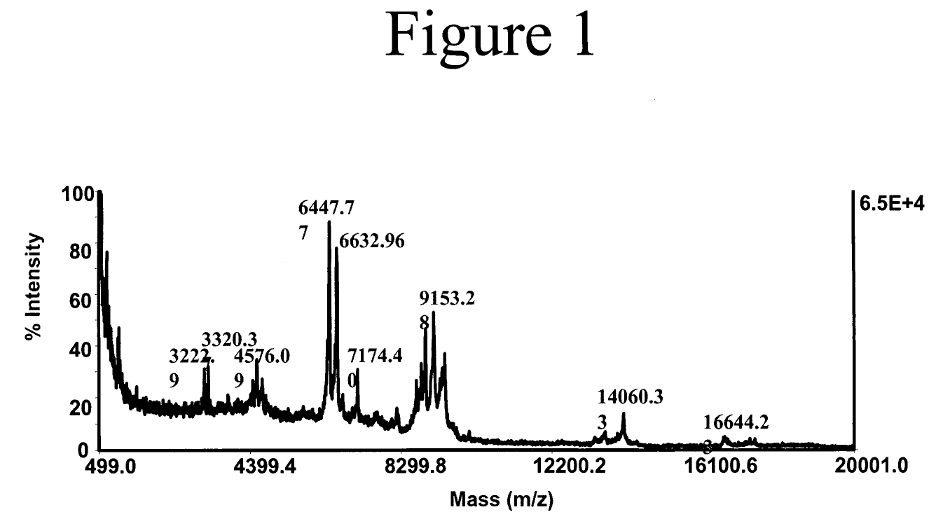

The pooled normal serum was diluted 1:10 in binding buffer containing saline (0.85%) to normalize the salt concentration, guanidine hydrochloride (1 M) to ensure protein denaturation and dissociation of protein-ligand complexes, and TFA (0.05%)+TBAP (2.5 mM) to improve polypeptide and protein adsorption of a final volume 25 μL of sample. The diluted serum sample in binding buffer was applied to a C4 solid phase ZipPlate prewetted with acetonitrile. Binding to the C4 solid phase was performed under low vacuum for a minimum of two minutes. The C4 solid phase resin was then washed 3 times with an aqueous solution containing 0.1% TFA under full vacuum to remove any unbound or weakly bound components of the serum solution. The bound analytes were then eluted under low vacuum (4 inches of Hg) with direct deposition onto a MALDI target using 1.5 μL of elution solvent composed of alpha cyano-4-hydroxy-cinnamic acid (CHCA, 5 mg/ml), acetonitrile (60% by volume), TFA (0.1% by volume), and ammonium phosphate (10 mM) in water and allowed to air dry. Linear mode MS analysis was performed using the Voyager DE-sSTR Workstation (ex Applied Biosystems). The collected spectrum is provided in FIG. 1.

### Example 2

### Preparation of a Pooled Normal Male Serum (Sigma H-3188) Using a C18 Solid Phase with Analysis by MALDI-TOF-MS.

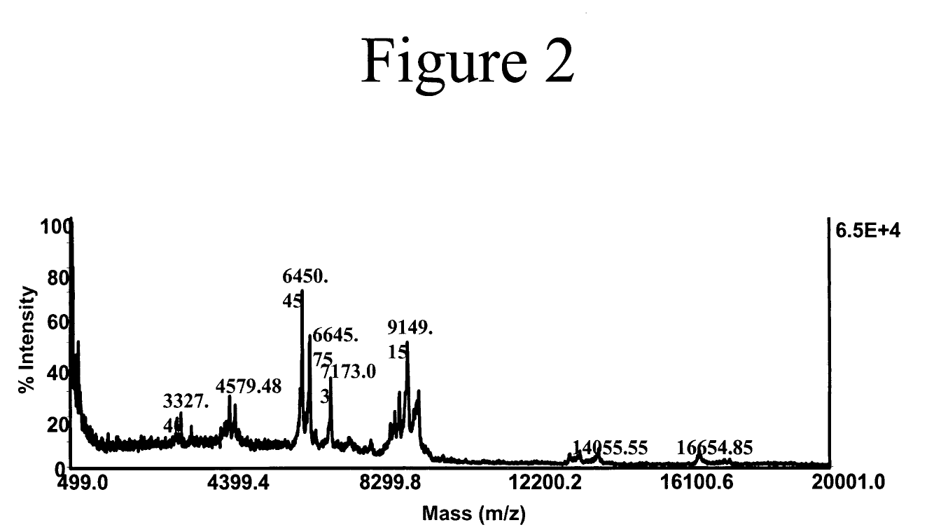

The pooled normal serum was diluted 1:10 in binding buffer containing saline (0.85%) to normalize the salt concentration, guanidine hydrochloride (1M) to ensure protein denaturation and dissociation of protein complexes, and TFA (0.05%) and TBAP (2.5 mM) to improve separation and acidify the solution for subsequent solid phase extraction for a final volume of 25 μL. The diluted serum sample was then applied to a C18 solid phase ZipPlate prewetted with acetonitrile. Binding to the C18 solid phase performed under low vacuum for a minimum of two minutes. The C18 solid phase resin was then washed 3 times with an aqueous solution containing 0.1% TFA solution under full vacuum to remove any unbound or weakly bound components of the serum solution. The bound analytes were then eluted under low vacuum (4 inches of Hg) with direct deposition onto a MALDI target using 1.5 μL of elution solvent composed of alpha cyano-4-hydroxy-cinnamic acid (CHCA, 5 mg/ml), acetonitrile (60% by volume), TFA (0.1% by volume), and ammonium phosphate (10 mM) in water and allowed to air dry. Linear mode MS analysis was performed using the Voyager DE-sSTR Workstation (ex Applied Biosystems). The collected spectrum is provided in FIG. 2.

### Example 3

### Preparation of a Low Molecular Weight Fraction of a Pooled Normal Male Serum (Sigma H-3188) Using a C4 Stationary Phase with Analysis by MALDI-TOF-MS

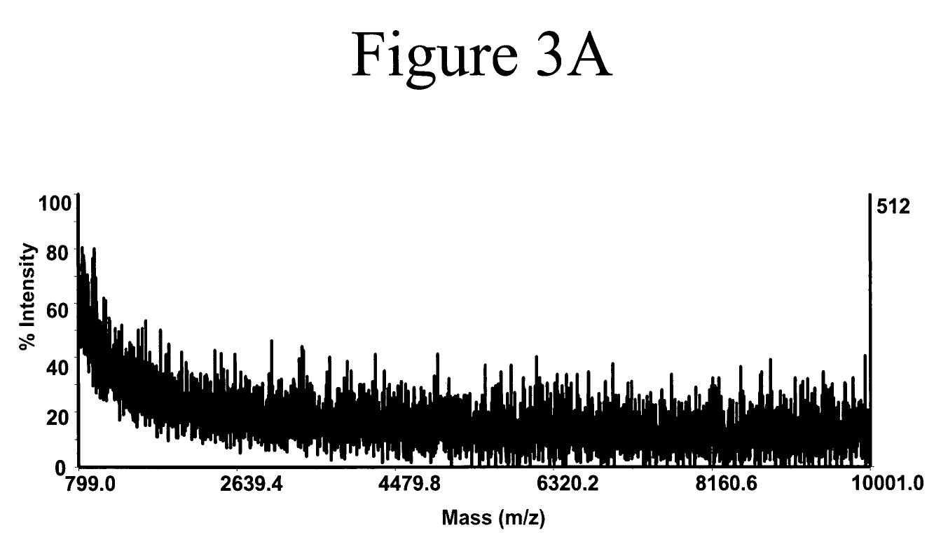

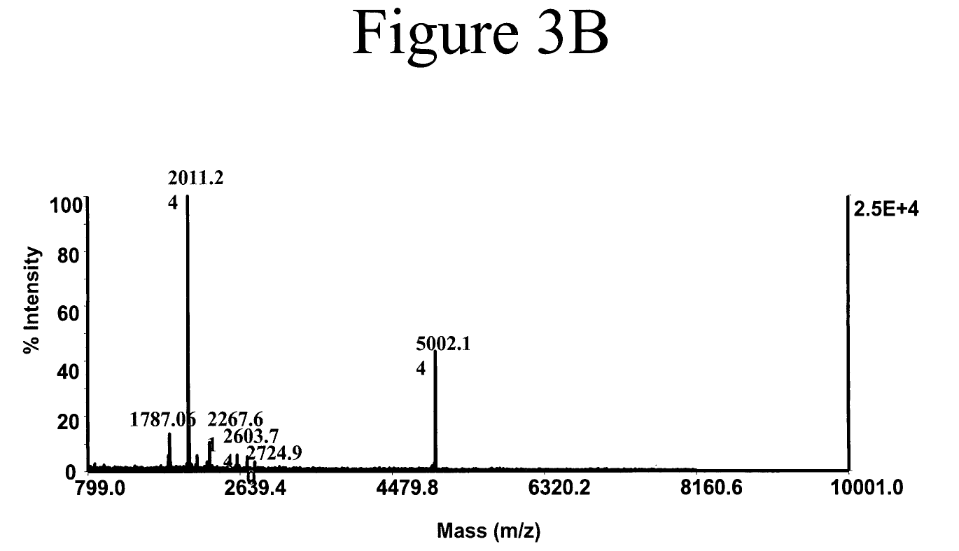

The pooled normal serum was diluted 1:10 for a final volume of 25 μL in binding buffer containing saline (0.85%) to normalize the salt concentration, guanidine hydrochloride (1M) to ensure protein denaturation and dissociation of protein complexes, and TFA (0.05%) and TBAP (2.5 mM) to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid phase extraction using a C4 ZipPlate as described in Example 1, and washed with a solution containing TFA (0.1% by volume) to remove the salts and other contaminants, which might interfere with analysis in the mass spectrometer. The bound analytes were eluted and directly deposited onto a MALDI target using an elution solvent composed of alpha cyano-4-hydroxy-cinnamic acid (CHCA, 5 mg/ml), acetonitrile (60% by volume), TFA (0.1% by volume), and ammonium phosphate (10 mM) in water and allowed to air dry. Reflector mode MS analysis was performed on the Voyager DE-sSTR Workstation (ex Applied Biosystems) on the serum sample both with and without the removal of the high molecular weight components for comparison. The collected spectrum is provided in FIG. 3b. For comparison FIG. 3a is a Reflector mode MALDI-MS spectrum without removal of high molecular weight components (i.e. prepared as described in Example 1). No signal could be generated from this sample and it was concluded that it was beneficial to remove such high molecular weight components prior to subsequent MALDI-MS data collection if Reflector mode operation was to be used for sample collection.

### Example 4

### Preparation of a Low Molecular Weight Fraction of a Pooled Normal Serum Using a C18 Stationary Phase with Analysis by MALDI-TOF-MS

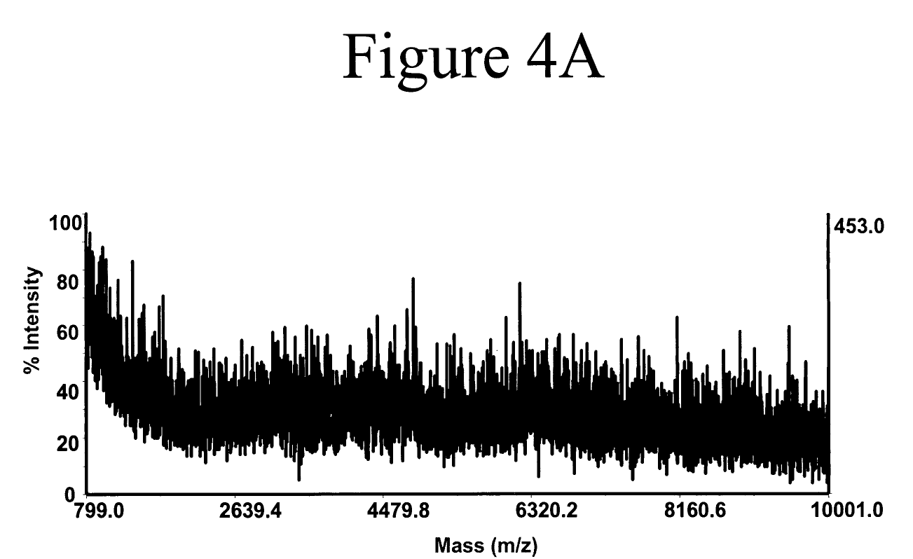

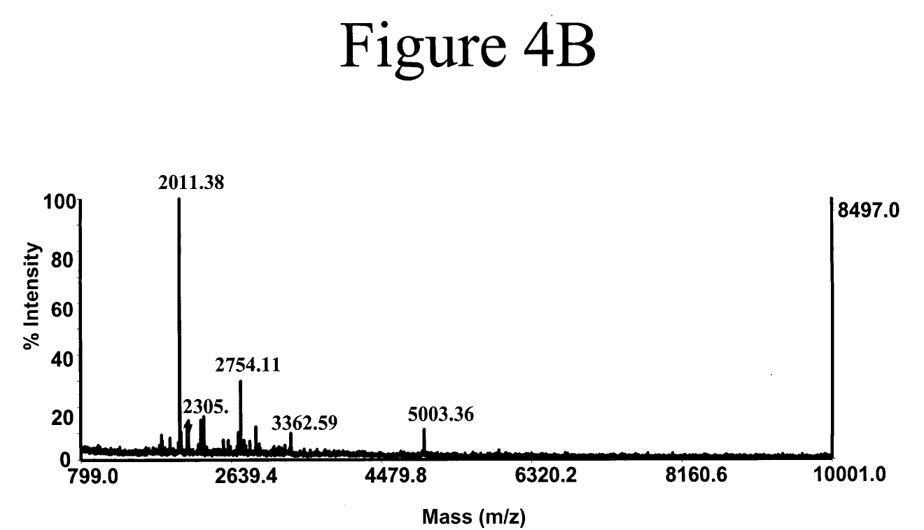

The pooled normal serum was diluted 1:10 for a final volume of 25 μL in binding buffer containing saline to normalize the salt concentration, a chaotropic agent to ensure protein denaturation and dissociation of protein complexes, and two ion pair reagents to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid phase extraction using a C18 ZipPlate as described in Example 2, and washed with a solution containing TFA (0.1% by volume) to remove the salts and other contaminants, which might interfere with analysis in the mass spectrometer. The bound analytes were eluted and directly deposited onto a MALDI target using an elution solvent composed of alpha cyano-4-hydroxy-cinnamic acid (CHCA, 5 mg/ml), acetonitrile (60% by volume), TFA (0.1% by volume), and an ammonium phosphate (10 mM) in water and allowed to air dry. Reflector mode MS analysis was performed using the Voyager DE-sSTR Workstation (ex Applied Biosystems) on the serum sample with and without the removal of the high molecular weight components for comparison. The collected spectrum is provided in FIG. 4b. For comparison, FIG. 4a is a Reflector mode MALDI-MS spectrum without removal of high molecular weight components (i.e. prepared only as described in Example 2). No signal could be generated from this sample and it was concluded that it was beneficial to remove such high molecular weight components prior to subsequent MALDI-MS data collection if Reflector mode operation was to be used for sample collection.

### Example 5

### Preparation of a Low Molecular Weight Fraction of a Pooled Normal Male Serum Sample

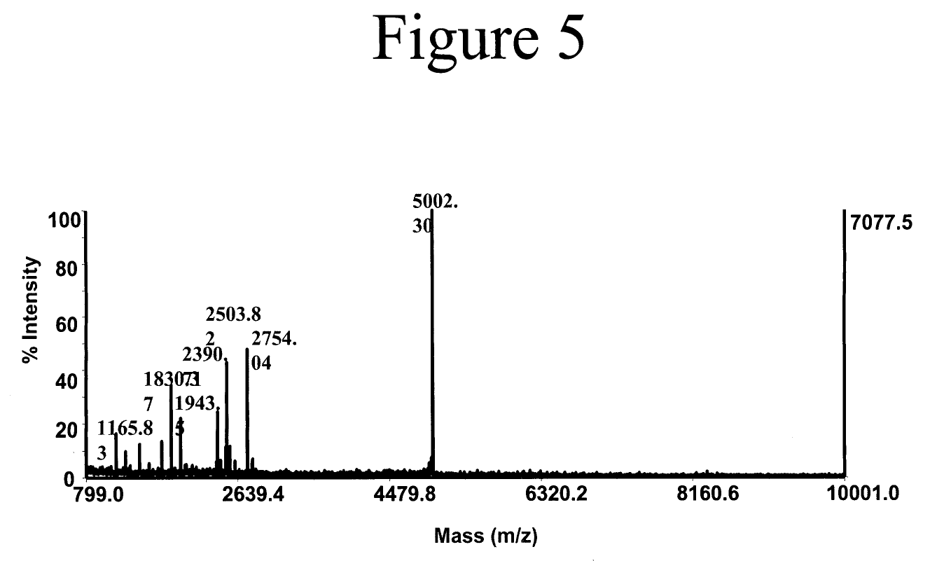

This example is similar to Example 3 using a C4 stationary phase with analysis by MALDI-TOF-MS, except from an alternative supply (Pierce ImmunoPure 31876). Like Example 3, the pooled normal serum was diluted 1:10 for a final volume of 25 μL in binding buffer containing saline (0.85%) to normalize the salt concentration, guanidine hydrochloride (1M) to ensure protein denaturation and dissociation of protein complexes, and TFA (0.05%) and TBAP (2.5 mM) to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid-phase extraction using a C4 ZipPlate as described in Example 1, and washed with a solution containing TFA (0.1% by volume) to remove the salts and other contaminants, which may interfere with analysis in the mass spectrometer. The bound analytes were eluted and directly deposited onto a MALDI target using an elution solvent composed of alpha cyano-5-hydroxy-cinnamic acid (CHCA, 5 mg/mL), acetonitrile (60% by volume), TFA (0.1% by volume), and an ammonium phosphate (10 mM) in water and allowed to air dry. Reflector mode MS analysis was performed on the Voyager DE-sSTR Workstation (ex Applied Biosystems). The collected spectrum is provided in FIG. 5. Comparison of this spectrum with that shown in FIG. 3B shows clear differences in the profile and peak intensities compared with the spectra presented in FIG. 3B, especially the significant increase of the peak at 5003 m/z. This was attributed to different processing methods used to prepare the sera, but demonstrates the utility of the described method for detection of differences between two unique sera.

### Example 6

### Fractionation of a Pooled Normal Male Serum Sample Using Ultrafiltration, with Detection by Reflector Mode MALDI-MS

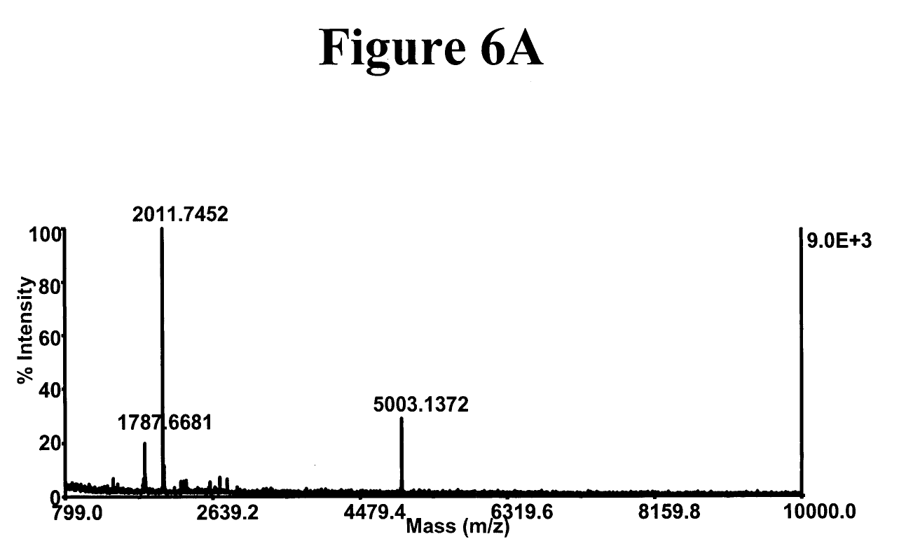

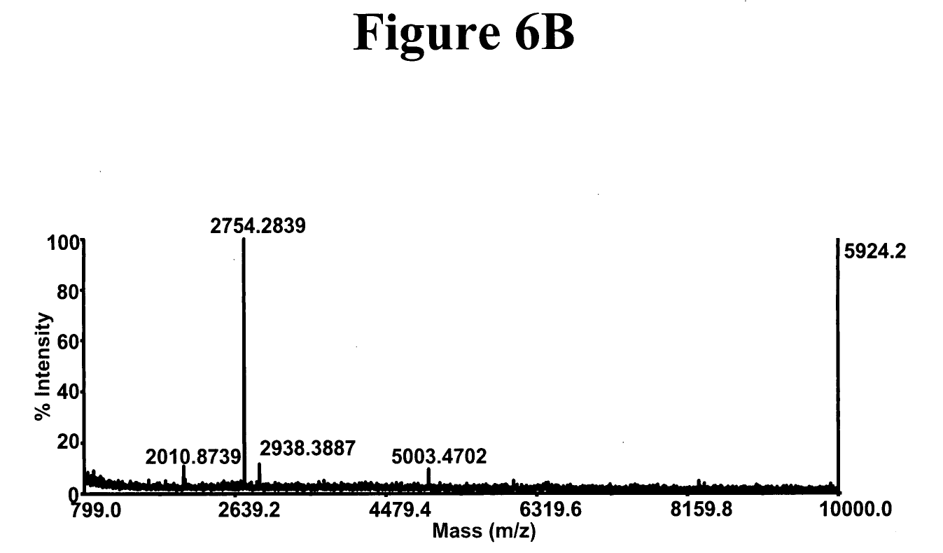

This example is similar to Example 3 using a C18 stationary phase with analysis by MALDI-TOF-MS, except following collection of a first fraction from the ultrafiltration device the material remaining in the device was incubated with a second buffer that contained a higher concentration of chaotropic agent. Like Example 3, the pooled normal serum was diluted 1:10 for a final volume of 25 μL in binding buffer containing saline (0.85%) to normalize the salt concentration, guanidine hydrochloride (1M) to ensure protein denaturation and dissociation of protein complexes, and TFA (0.05%) and TBAP (2.5 mM) to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid phase extraction using a C18 ZipPlate as described in Example 1, and washed with a solution containing TFA (0.1% by volume) to remove the salts and other contaminants, which may interfere with analysis in the mass spectrometer. The bound analytes were eluted and directly deposited onto a MALDI target using an elution solvent composed of alpha cyano-5-hydroxy-cinnamic acid (CHCA, 5 mg/mL), acetonitrile (60% by volume), TFA (0.1% by volume), and an ammonium phosphate (10 mM) in water and allowed to air dry. Reflector mode MS analysis was performed on the Voyager DE-Pro Workstation (ex Applied Biosystems). The collected spectrum is provided in FIG. 6A. The ultrafiltration device was retained and treated with a second buffer solution containing saline (0.85%) to normalize the salt concentration, guanidine hydrochloride (2M) to aid further dissociation of protein complexes and aggregates, and TFA (0.05%) and TBAP (2.5 mM). Subsequently this solution was collected by centrifugation and passed through an unused well of a ZipPlate. Washing of the ZipPlate and elution steps were as described above. This second fraction was analyzed by Reflector mode MS analysis using a Voyager DE-Pro Workstation (ex Applied Biosystems). The collected spectrum is provided in FIG. 6B. Clear differences the collected Reflector mode spectra provided in FIGS. 6A and 6B were seen and prove fractionation of the serum by this approach.

### Example 7

### Fractionation of a Pooled Normal Male Serum Sample Using Ultrafiltration with Detection by Linear Mode MALDI-MS

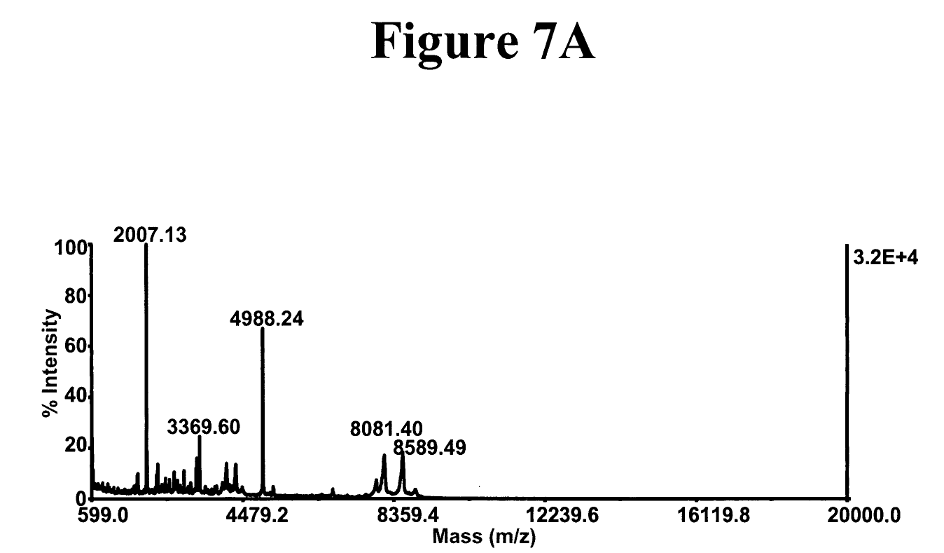

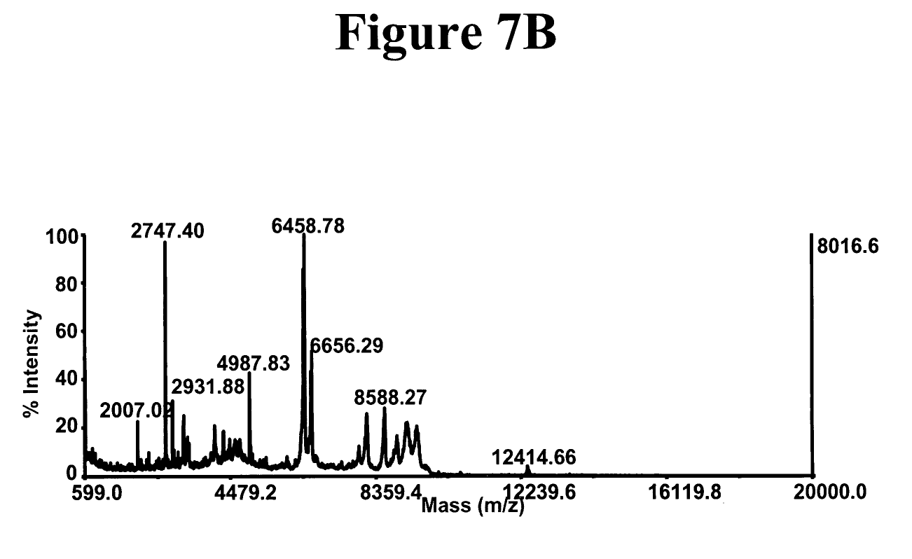

This example is similar to Example 3 using a C18 stationary phase with analysis by MALDI-TOF-MS, except following collection of a first fraction from the ultrafiltration device the material remaining in the device was incubated with a second buffer that contained a higher concentration of chaotropic agent. Like Example 3, the pooled normal serum was diluted 1:10 for a final volume of 25 μL in binding buffer containing saline (0.85%) to normalize the salt concentration, guanidine hydrochloride (1M) to ensure protein denaturation and dissociation of protein complexes, and TFA (0.05%) and TBAP (2.5 mM) to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid phase extraction using a C18 ZipPlate as described in Example 1, and washed with a solution containing TFA (0.1% by volume) to remove the salts and other contaminants, which may interfere with analysis in the mass spectrometer. The bound analytes were eluted and directly deposited onto a MALDI target using an elution solvent composed of alpha cyano-5-hydroxy-cinnamic acid (CHCA, 5 mg/mL), acetonitrile (60% by volume), TFA (0.1% by volume), and an ammonium phosphate (10 mM) in water and allowed to air dry. Linear mode MS analysis was performed on the Voyager DE-Pro Workstation (ex Applied Biosystems). The collected spectrum is provided in FIG. 7A. The ultrafiltration device was retained and treated with a second buffer solution containing saline (0.85%) to normalize the salt concentration, guanidine hydrochloride (2M) to aid further dissociation of protein complexes and aggregates, and TFA (0.05%) and TBAP (2.5 mM). Subsequently this solution was collected by centrifugation and passed through an unused well of a ZipPlate. Washing of the ZipPlate and elution steps were as described above. This second fraction was analyzed by Linear mode MS analysis using a Voyager DE-Pro Workstation (ex Applied Biosystems). The collected spectrum is provided in FIG. 7B. Clear differences the collected Reflector mode spectra provided in FIGS. 7A and 7B were seen and prove fractionation of the serum by this approach.

### Example 8

### Identification of Components of a Pooled Normal Male Serum Sample by MALDI-MS/MS After Sample Preparation Using Ultrafiltration, and Solid Phase Extraction

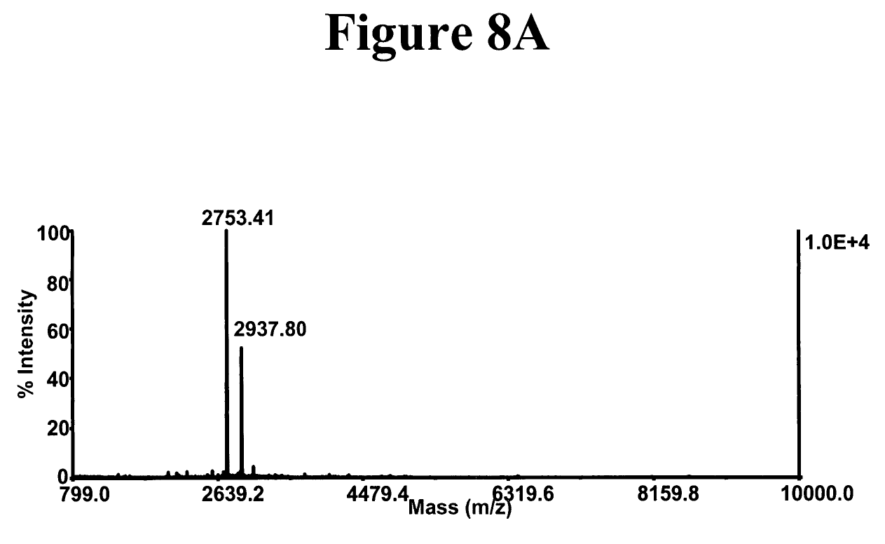

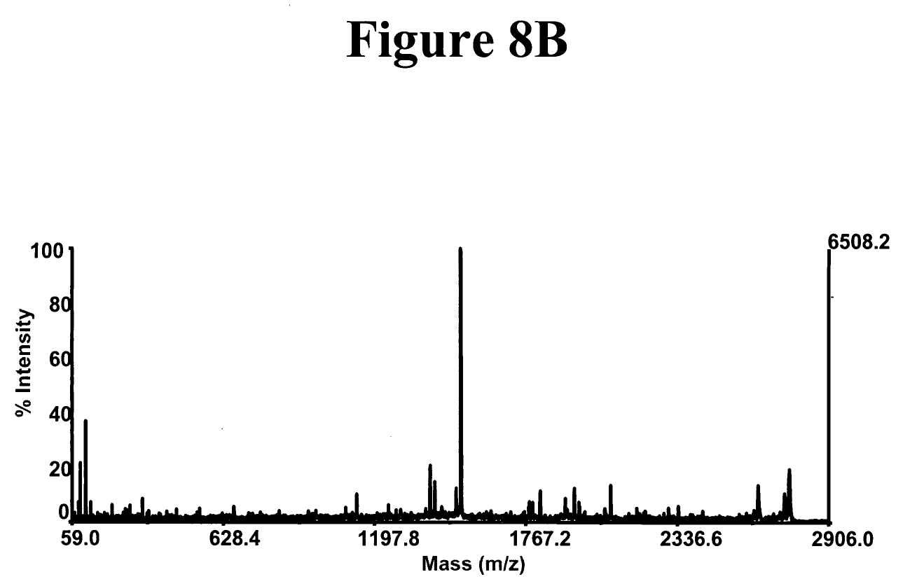

This example is similar to Example 3 using a C18 stationary phase with analysis by MALDI-TOF-MS, except following collection of a first fraction from the ultrafiltration device the material remaining in the device was incubated with a second buffer that contained a higher concentration of chaotropic agent. Like Example 3, the pooled normal serum was diluted 1:10 for a final volume of 25 μL in binding buffer containing saline (0.85%) to normalize the salt concentration, guanidine hydrochloride (2M) to ensure protein denaturation and dissociation of protein complexes, and TFA (0.05%) and TBAP (2.5 mM) to improve separation and acidify the solution for subsequent solid phase extraction. The diluted serum solution was ultrafiltered using a Microcon centrifugal filter device from Millipore, 50,000 molecular weight cut off. The low molecular weight filtrate was then fractionated by solid phase extraction using a C18 ZipPlate as described in Example 1, and washed with a solution containing TFA (0.1% by volume) to remove the salts and other contaminants, which may interfere with analysis in the mass spectrometer. The bound analytes were eluted and directly deposited onto a MALDI target using an elution solvent composed of alpha cyano-5-hydroxy-cinnamic acid (CHCA, 5 mg/mL), acetonitrile (60% by volume), TFA (0.1% by volume), and an ammonium phosphate (10 mM) in water and allowed to air dry. Reflector mode MS analysis was performed on a 4700 Proteomics Discovery System (ex Applied Biosystems). The collected spectrum is provided in FIG. 8A. From this spectrum ions were chosen for subsequent analysis by MS/MS using the 4700 Proteomics Discovery System (ex Applied Biosystems). In this example the response detected at a mass to charge ratio (m/z) of 2753 was selected and fragmented to produce a MS spectrum, provided in FIG. 8B that was characteristic of a fragmented peptide. The amino acid sequence of this peptide was determined by a database search using the GPS Explorer software package (ex Applied Biosystems), and was determined to be from albumin. This example also demonstrates the different profiles that may be generated when using binding buffers of different composition. The Reflector spectra provided in FIGS. 6A and 8A display clear differences. This was the same sample prepared with a binding buffer that contained either 1M guanidine hydrochloride (saline, TBAP, and TFA), FIG. 6A, or separately prepared with a binding buffer that contained 2M guanidine hydrochloride (saline, TBAP, and TFA), FIG. 8A.

1. A kit comprising one or more binding buffers, each binding buffer comprising:
a) salt concentration in the range of about 0.1% to about 2.0%;
b) at least one chaotropic agent;
c) at least one reducing agent; and
d) at least one volatile organic acid.

2. The kit of claim 1, further comprising:
e) at least one strong ion pair reagent.

3. The kit of claim 1, further comprising one or more ultrafiltration devices.

4. The kit of claim 1, further comprising one or more solid phases.

5. The kit of claim 1, further comprising one or more elution solvents.

6. The kit of claim 2, wherein the at least one strong ion pair reagent is heptafluorobutyric acid, tetrabutylammonium phosphate (TBAP) or triethylamine trifluoracetic acid.

7. The kit of claim 6, wherein the at least one strong ion pair reagent is present in the range of about 0.5 mM to about 100 mM in the binding buffer.

8. The kit of claim 1, wherein the at least one chaotropic agent is present in the range of about 0.01M to about 8 M in the binding buffer.

9. The kit of claim 1, wherein the at least one reducing agent is present in the range of about 0.01 mM to about 100 mM in the binding buffer.

10. The kit of claim 1, wherein the at least one volatile organic acid is present in the range of about 0.001% to about 5% v/v in the binding buffer.

11. A method comprising:
a) diluting a sample of biological fluid with a binding buffer, wherein the binding buffer comprises:
i) salt concentration in the range of about 0.1% to about 2.0%;
ii) at least one chaotropic agent;
iii) at least one reducing agent; and
iv) at least one volatile organic acid;
b) contacting the diluted sample with a solid phase device to thereby immobilize one or more components of the sample on the solid phase;
c) washing the solid phase with a wash solvent;
d) eluting one or more of the components from the solid phase device with one or more elution solvents; and
e) collecting one or more fractions of eluent from the solid phase device.

12. The method of claim 11, further comprising:
f) analyzing the one or more fractions of eluent by mass spectrometry.

13. The method of claim 11, further comprising:
f) treating the biological sample with at least one ultrafiltration device prior to performing step (a).

14. The method of claim 11, further comprising:
f) treating the biological sample with at least one ultrafiltration device prior to performing step (b).

15. The method of claim 12, further comprising:
f) treating the biological sample with at least one ultrafiltration device prior to performing step (f).

16. The method of claim 11, wherein only one fraction of eluent is collected from the solid phase device.

17. The method of claim 11, wherein the components bound to the solid phase device are fractionated between two or more different fractions.

18. The method of claim 11, wherein the binding buffer further comprises:
v) at least one strong ion pair reagent.

19. The method of claim 18, wherein the at least one strong ion pair reagent is heptafluorobutyric acid, tetrabutylammonium phosphate (TBAP) or triethylamine trifluoracetic acid.

20. The method of claim 19, wherein the at least one strong ion pair reagent is present in the range of about 0.5 mM to about 100 mM in the binding buffer.

21. The method of claim 11, wherein the at least one chaotropic agent is present in the range of about 0.01M to about 8 M in the binding buffer.

22. The method of claim 11, wherein the at least one reducing agent is present in the range of about 0.01 mM to about 100 mM in the binding buffer.

23. The method of claim 11, wherein the at least one volatile organic acid is present in the range of about 0.001% to about 5% v/v in the binding buffer.
